SEQUENCE LISTING (10 sequences)

SEQ ID NO: 1 - DNA, 20 bp, Artificial Sequence
  SEQ ID No. 1 Primer for Real-time-PCR, forward primer, determination of MAP ISMAv2-sequence, bindingposition 1645 to 1665 at the ISMav2-sequence
cggcaaaatcgagcagtttc

SEQ ID NO: 2 - DNA, 21 bp, Artificial Sequence
  SEQ ID No. 2 Primer for Real-time-PCR, reverse primer, Identification of MAP ISMav2-sequence, bindingposition 1786 to 1807 at the ISMav2-sequence
tgagccggtgtgatcatcttt

SEQ ID NO: 3 - DNA, 15 bp, Artificial Sequence
  SEQ ID No. 3 TaqManmgb-probe ISMav2, identification of MAP ISMav2-sequence, bindingposition 1678 to 1693 at the ISMav2-sequence
cgctgagttccttag

SEQ ID NO: 4 - DNA, 15 bp, Artificial Sequence
  SEQ ID No. 4 LNA-probe ISMav2, identification of MAP ISMav2 sequence, bindingposition 1678 to 1693 at the ISMav2-sequence
cgctgagttccttag

SEQ ID NO: 5 - DNA, 19 bp, Artificial Sequence
  SEQ ID No. 5 Primer for Real-time-PCR, forward primer F57, identification of  MAP F57 sequence, bindingposition 244 to 263 at the F57-sequence
tacgagcacgcaggcattc

SEQ ID NO: 6 - DNA, 19 bp, Artificial Sequence
  SEQ ID No. 6 Primer for Real-time-PCR, reverse primer F 57, identification of MAP F57 sequence, bindingposition 288 to 307 at the F57-sequence
cggtccagttcgctgtcat

SEQ ID NO: 7 - DNA, 14 bp, Artificial Sequence
  SEQ ID No. 7 TaqManmgb-probe F57, identification of MAP 57 sequence, bindingposition 268 to 282 at the F57-sequence
cctgaccacccttc

SEQ ID NO: 8 - DNA, 14 bp, Artificial Sequence
  SEQ ID No. 8 LNA-probe F57, identification of MAP F57 sequence, bindingposition 268 to 282 at the F57-sequence
cctgaccacccttc

SEQ ID NO: 9 - DNA, 2263 bp, Mycobacterium avium
  gene (1)..(2263): ISMav2 putative transposase gene, sequence derived from Mycobacterium aviom ssp. paratuberculosis (MAP)
gagctcggccagcacttcatccgggtgaattccgtgcaccccaccaacgtgaacacaccgatgttcatgaacgaggggacgatgaggctgttccggccggacctgaagaaccccggcccggacgacctgaaggtcgccgcgcagttcatgcatgtgctgccggtcggctgggtggagccggtggacatcagcaacgccgtgctgttcctggcctccgacgaatcgcgttacatcacaggtcttccggtcaccctcgacgccggcagcatgctcaagtagccgcgccggcatctcgctcaccggcggtgaggatcatcccggtggccaggggcgggatcgaaccgccgaccttccgcttttcaggcggacgctcgtaccgactgagctacctggccggaaggcagcgagtggctcgccgcgctggcgaccctgacgggactcgaacccgcgacctccgccgtgacagggcggcgcgctaaccaactgcgccacagggccttgctgctgctccgcgtcgccgcgttgcgtaccccctacgggattcgaacccgcgctaccgccttgaaagggcggcgtcctaggccgctagacgaagggggccagaaccgaatctctccggggtactcgcaacgtggtttcgttgggagccacgtcagcttaggtcaccgtgggcccaatcctcaaacgagcccggttttgggtccaagtatcctgaaactccgcggcccctatagctcagttggtagagctacggacttttaatccgcaggtcctaggttcgagtcctagtgggggcaccagatgtatgtatgacgtcggctgatgtgtgacgttttgtcttcggttgatgtgtgacggtgtttcggttgatgcgtgacagttgtttcggctgatcggtgacactccctagatgagggagttaagtgtggctgagcagcggtatcaggccgtgatggcggtaatcagtgacgggttgtcggtgtcgcaggccgcagagaagttcggggtggcgcgtcagacgctgcaccgatggctggcccggtatgaagccgcgggcctgaaggggctggtggatcggtcgcatcggccggtgagttgtccgcatcagatgctggcggtagtggaggcggcggtgttggagttgcgccgctcgcggccctattgggggccgcggcggttggtgttcgagttggccaagcgaggtgtccatccggtgccgtcggagtcgccggtgtatcgagcgctggtgcgggccggtctgatcgaccccgcgatgcgagatcgacggtcgcgcaaatggaagcgctgggagcgcggggcgccgatggagttgtggcagctcgacatcgtcggcgggttcccgctggccgatgggaccagcgccaaagccctgaccggcatcgatgatcattcccggatgtgtgtgtgcgccaagctgatggcccgtgagcgcacccgcgcggtctgcgacggattacgggcggcgctggccgcttacggggtgcctgagcagatcttgaccgataacggcaaggtgttcaccgagcggttttgtcatccaccggtcgaggtgctctttgatgcgatctgccgcgagcacggcatcgaacatctgttgacccagccgcgcagcccgaccacgaccggcaaaatcgagcagtttcaccgcagtctgcgcgctgagttccttagcggccgtgagcctttcaccaacctcaaggtcgctcagcaggcgctcgatgagtgggtcgaggactacaacaccacccggccgcaccaagccctaaagatgatcacaccggctcaacggtttcacgccggtgcgccggcatcaccaccgtcgaactcgtgtgcccgacacgtcgaccgcagtggtgatgactgggtgagtcggcgggtgtgctccaacggcatcgtgtgcgtgtcctggcagcaggtctgtatcgggcgccactatgccggcgcccgctgcgatgtccacgtcgatggggacctgctgaggttttgggtcggcgacaatctggtcaaaaccgccgcgcgcaccagccgcggcgaggtacgaaacaaacaggccctgcgcaccaacgcaccggcctaaaacacaacccagagtgtcaccgatcaaccgacatagaaatgtcaccgagcaaccgaccctgaacaggggcaccagagagcagccccgcccgctcaggtggtcgacgatgtggctgcgctcgccgtggtagaccaggtcattgaaatcgacctcgaacccgagctc

SEQ ID NO: 10 - DNA, 620 bp, Mycobacterium avium
  misc_feature (1)..(620): Mycobacterium avium ssp. paratuberculosis species specific F57 DNA fragment
ggatctcggccccgatagctttcctctccttcgtcaccaactggcgcgggtccaggaacgcttggcactcgtcaatcaccacgaacaccagcggaacctgcggcgtcgggccgcgatcccaaaagttggacgatccgaatatgttgttgccctgtctaattcgatcacggactagaccggtcgcgtcattcagaatcgctgcaatctcaggcagctccagatcgtcattcatgaatcggcagctacgagcacgcaggcattccaagtcctgaccacccttcccgtcgatgacagcgaactggaccgccgctgacgcaccgaacgaccccagagcactcgtcagccacgcggttttccccgaccctggtacgcctcccactacaacgcccgatacgttcgccaacgggagcgactggtagacgcccatttcatcgatacccaaactcagagaccacgagtgggcggctggcacagacgaccattcaagcggcgaactaagcggatcgacaattctcagctgcaactcgaacacacctgggaccggctcggtgacaacaacattcggtacaccaatagctgacgccagcttcggagccgccacctgccactgtctgagatcc

(19) United States
(12) Patent Application Publication
(10) Pub. No.: US 2010/0196894 A1
(43) Pub. Date: Aug. 5, 2010

(54) PROCEDURE FOR THE DETECTION OF PARATUBERCULOSIS

(76) Inventors: Michael Buelte, Reinhardshains (DE); Amir Abdulmawjood, Giessen (DE); Holger Schonenbrucher, Grossosteheim-Pflaumheim (DE)

Correspondence Address: CLARK & BRODY, 1700 Diagonal Road, Suite 510, Alexandria, VA, 22314, US

(21) Appl. No.: 12/593,703

(22) PCT Filed: Mar. 29, 2008
(86) PCT No.: PCT/DE2008/000523

§ 371 Date: Mar. 26, 2010

(30) Foreign Application Priority Data
Mar. 30, 2007 (DE) 10 2007 015 775.6

(51) Int. Cl. C12Q 1/68; C07H 21/04

(52) U.S. Cl. 435/6; 536/24.33

(57) ABSTRACT

The present invention relates to a procedure for the specific and sensitive detection of Mycobacterium avium ssp. paratuberculosis (MAP) in fecal, tissue and organ samples using a real-time PCR procedure.

The present invention relates to a procedure for the specific and sensitive detection of Mycobacterium avium ssp. paratuberculosis (MAP) in organ, tissue and fecal samples using a real-time PCR procedure with genomic deoxyribonucleic acid (DNA) of a suitable marker.

### STATE OF THE ART

Mycobacterium avium ssp. paratuberculosis (MAP) was described 1895 for the first time as etiological agent for a chronic granulomatous enteritis in a cow in Germany. After the discoverer, the name Johne's Disease was introduced for this in ruminants but also other mammals occurring paratuberculosis (ParaTB). It is a disease which is spread almost world-wide and causes considerable economic losses.

Due to the comparable pathomorphological changes of the chronic inflammatory intestine disease which is called Morbus Crohn (MC) in humans, it was suspected already at the beginning of the last century that mycobacteria like these might play a role as etiologic agent in the infectious process. The name is derived from a description of eight cases of ileitis by Crohn, Ginzburg, and Oppenheimer (1932) at the Mount Sinai Hospital in New York. The first precise characterization of the clinical course was already carried out 1913 by Dalziel in Glasgow. The inflammatory enteritis proceeds in intervals and implies a substantial reduction of the quality of life for persons affected. The lethality rate is in the range of approx. 6%. A multi-center study for Europe indicates that the incidence, i.e. the number of new cases of MC amounts to 5.6 cases per 100,000 inhabitants per year; thus approx. 200,000 people are affected.

The prevalence of MC in Germany was estimated to range from 1/500 to 1/800, which corresponds to about 200,000 patients. The incidence amounts to 5.2 per 100,000 inhabitants. Thereby, the total expenses are reported to be 20,000 [symbol] /year and patient, adding up to an amount of approx. 2 bn per year in Germany. The similarities between bovine ParaTB and the human intestinal TB and MC with respect to clinical and histological appearance cannot be denied, the more so as MAP could be isolated from tissue samples of patients. With the improvement of detection methods and the introduction of PCR methodology, MAP could be confirmed more and more often in sick animals and people.

For the molecular biological diagnostics, the insertion element “IS900” of the MAP-genome has long been acknowledged as standard. Thereby, scientific studies are exclusively based on the use of 5 different primer pairs. These are in detail IS900/150C//IS900/921, p36//p1, MK5/MK6, P90//P91, and P21/P8. However, cross-reactions with so-called “IS900like”-elements of other organisms have also been reported, which render this marker inapplicable for the specific detection of

MAP. Several prior art studies nevertheless describe the development of IS900-based real-time PCR-procedures. These methods combine oligonucleotides which are used in conventional PCR-procedures with markers which are well-established in ELISA-techniques, e.g. fluorescent dyes or radioactive markers such as e.g. disclosed in DE 102 24 338 A1 for the detection of MAP. However, these methods do not provide the specificity which is required for the reliable detection of MAP, and are susceptible for contaminations or false-positive test results.

From scientific studies, a further marker for MAP is known, the insertion sequence ISMav2 of the MAP-genome. ISMav2 shares in fact no similarities with other mycobacterial insertion sequences, but shares 50% identity with a potential transposon of Streptomyces coelicolor, so that possible cross-reactions during the analysis of biological samples can also not be excluded here.

Furthermore it is known from DE 10 2005 001 788 A1, that the F57-sequence of the MAP-genome is a highly specific marker for MAP and therefore represents a suitable target structure for selective PCR detection methods.

DE 696 27 625 T2 discloses the dnaJ-gene, a gene of the heat shock protein, for the detection and determination of mycobacteria.

Due to cross-reactions however are F57 and dnaJ marker sequences alone also not suitable for a use in real-time PCR procedures for the specific and reliable detection of MAP in biological samples.

A further disadvantage of state of the art techniques is that the detection of MAP is mainly performed using milk, as disclosed e.g. in DE 102 24 338 A1. Milk represents a biological sample which can very easily be analyzed, containing only very few contaminating microorganisms and no substances which interfere with PCR reactions. Thus, no particular requirements have to be met during sample preparation and procedure of the PCR reaction.

However, the analysis of other biological samples such as fecal, organ and tissue samples is required, because not all live stocks (including humans) which have to be surveyed routinely as potential center of an epidemic for paratuberculosis give milk.

A person skilled in the art knows that the analysis of complex biological samples makes high demands and that a procedure which is well-established for milk analysis cannot be transferred directly.

The problem of cross-reactions, neutralizing, and the general contamination with other microorganisms is particularly high in fecal samples which have a complex composition. A further enrichment of the pathogen in a culture medium is generally undesirable, since a fast diagnosis has to be guaranteed and the cultivation of MAP takes a long time.

To date, no state-of-the-art procedure is known which, using real-time PCR, satisfactorily discloses the automated, specific, and reliable detection of MAP in biological samples, most notably in fecal, organ and tissue samples.

### Aim

The aim of the present invention is therefore to eliminate the disadvantages of state-of-the-art techniques as described and to provide an improved real-time PCR procedure for the specific and reliable detection of MAP in fecal, organ and tissue samples, in addition to a test kit which is suitable for an automated processing of this method.

The aim of the present invention is solved by a procedure according to claims 2-10 and a test kit according to claims 11-26, using specific oligonucleotides according to claim 1.

The procedure according to the present invention offers numerous advantages which are connected with the use of real-time PCR, e.g. the application of a specific and reliable DNA extraction procedure for fecal, organ and tissue samples, which can be conducted as automated procedure with high sample throughput. False-negative results or cross-reactions can almost be excluded by the optimal choice of oligonucleotides as primers and the simultaneous amplification of two marker structures in the MAP gene. In addition, the comparison with an “internal amplification control (IAK)” during the real-time PCR allows an exact quantification of the amplified specific MAP-DNA. Consequently, an improved specific and at the same time sensitive real-time PCR procedure for the detection of Mycobacterium avium ssp. paratuberculosis (MAP) is provided.

According to the present invention, optimal primers are provided and used in combination for the amplification of marker sequences “ISMav2” and “F57” of the MAP-genome in a real-time procedure for the specific detection of genomic MAP-DNA in fecal, organ and tissue samples.

Thereby, the previous preparation of the biological material is of particular relevance.

Furthermore, an accordant test kit is provided for routine and automated application of the method as high-throughput procedure, which includes the optimal primers and alternatively also the internal amplification control (IAK) or materials for sample preparation and performance of the PCR-procedure, respectively.

The real-time PCR procedure according to the present invention detects selectively the existence of MAP-DNA in fecal, organ and tissue samples, independently of the animal species which is investigated. The procedure allows in addition the quantitative determination of MAP-DNA detected in the biological sample in the same process. Thereby, the specific detection occurs independently of the presence of concomitant flora in the biological sample as well as the concentration of known PRC-inhibitors in the sample. The MAP-detection is independent of the animal species, i.e. that as well MAP-strains of bovine, human, and ovine origin can be detected reliably.

Due to the high sensitivity of this method, an early detection is guaranteed, even at very low MAP concentrations in the biological sample, and at a very early time point, actions can be taken which make a substantial contribution to protect the health of people, but also animal health in live stocks and the quality of foods.

In comparison to conventional PCR-procedures, the real-time PCR-procedure according to the present invention is considerably more sensitive. For example, in cattle fecal samples, up to 10² colony forming units of MAP can be detected. The detection limit of the real-time PCR system, which is calculated according to a method which is commonly used by experts skilled in the art via an analysis of decadal dilution series, is in the range of approx. 1.0×10⁻¹² g/DNA per PCR. Thereby, particularly the possible inhibitory effect of the background-DNA in fecal, organ and tissue samples was inspected and no negative effects on the detection limit were observed.

The main advantage or the PRC-procedure according to the present invention is a by a factor of 30 increased sensitivity as compared to conventional PCR and the direct quantification which allows a qualified analysis. For the expert skilled in the art it is also evident that, due to the time-resolved and parallel measurements, the labor and time consuming gelelectrophoretic analyses are no longer required. Conventional PCR-procedures achieve a comparable sensitivity only when using the “nested PCR”. This method is however, as those skilled in the art know, inapt due to the high danger of contamination and the high rate of false-positive results associated with this.

Independently of the sample matrix and the particular degree of processing or heating, respectively, the reliable verification in combination with the sample preparation is obtained in less than 8 hours (“one day result”).

Together with the sample preparation procedures which were optimized and refined for this application in a combination as described here, a reliable and efficient procedure is provided which can also be conducted and easily evaluated by less skilled laboratory personnel. The procedural steps are also carried out in an automated manner, allowing a high sample throughput.

The procedure according to the present invention for the specific detection of Mycobacterium avium ssp. paratuberculosis (MAP) in fecal, organ and tissue samples using real-time PCR is characterized by the preparation of the sample material, the DNA extraction, and an analysis of isolated DNA in a real-time PCR, whereby both marker sequences ISMav2 and F57 of the MAP-genome are amplifled with suitable oligonucleotides as primers and detected with suitable probes.

The sample preparation is chosen in a way that total genomic DNA can be isolated even from complex biological samples such as fecal, organ and tissue samples after reducing the amount of inhibitors of the PCR-reaction, a mechanical homogenization, and a reliable proteinase K-treatment.

As MAP-specific markers according to the present invention, the marker sequences “ISMav2” and “F57” of MAP-genome are used, suitable oligonucleotides as forward and reverse primers for hybridization are synthesized on the basis of these gene sequences, and fluorescent probes which are appropriate for these primers are chosen.

The oligonucleotides used as primers are compatible with common probes of various manufacturers (e.g. TaqMan, LNA), which bind and hybridize to the amplified DNA, respectively, allowing to perform the real-time PCR according to the present invention independently of the equipment and with broad applicability.

The gene segment which is preferably chosen for the detection of MAP in biological samples using real-time PCR with primers according to the present invention has only a very small size, is evidently stable over several years in fecal, organ and tissue samples, and can reliably be detected even after a longer time.

### Embodiments

a) Preparation of Sample Material and DNA-Extraction

As sample material, i.e. the sample to be investigated or a positive or negative control sample, fecal, organ and tissue samples of humans, animals as for example cattle, poultry, sheep or further farm or domestic animals are used, among these also samples deriving from lymph nodes or intestinal parts. Due to the complexity of the samples to be investigated, the inhomogeneous distribution of MAP in the sample (agglutination), and the high stability of the MAP cell wall, the homogenization step is of importance and is thus optimized in order to avoid contaminations.

Among numerous standard procedures known to the expert for the homogenization of biological samples such as the use of a swing mill, the Elvehjem-Potter, the Ultra Turrax T25, preferably the highly efficient and time-saving homogenization with Fast-Prep-120 is performed. The homogenization step comprises here a combination of vertical rotary motion and horizontal up and down movements.

Since the sample material, especially feces, contains a high amount of inhibitors of the PCR reaction (e.g. bile acids, hemoglobin), particular demands are made on the DNA extraction.

The extraction of genomic DNA from fecal, organ and tissue samples is preferably performed after an efficient reduction of the amount of PCR inhibitors, the use of mechanical homogenization, and a reliable proteinase K-treatment. For the analysis of fecal samples, e.g. DNeasy® Stool mini Kit from Qiagen, for the analysis of organ and tissue samples e.g. DNeasy Tissue Kit from Qiagen according to the instructions of the manufacturer is used. In order to reduce the foam formation occurring here, a detergent e.g. DX-buffer is added. Cell disruption and DNA isolation are performed in an automated mode.

For example, sample preparation and DNA extraction from fecal samples are performed under the following conditions:

Working solution of DX- (Qiagen, Hilden) and ASL-buffer are prepared according to the instructions of the manufacturer (Stool Kit): for this purpose, 140 ml Qiagen buffer ASL are pre-heated to 70° C. and 350 μl DX-buffer are added and mixed. The DX-buffer reduces foam formation during the mechanical homogenization. Subsequently, 1 g feces each are given to 5 ml DX/ASL buffer mixture and mixed on a vortex. The suspension is incubated in a water bath for 10 min at 95° C. 1.3 ml of the supernatant are each transferred into a 2 ml plastic tube with screw cap, which was previously equipped with 4 glass beads. The mechanical disruption is performed e.g. in a ribolyzer according to the following procedure: 4×20 sec at level 6, followed by mixing on a vortex for 15 s. The supernatant is quantitatively removed and transferred into a new 2 ml tube. To achieve a distinct separation of solid phase and supernatant, a centrifugation is carried out at 5000×g/5 min. After addition of one InhibitEX tablet per sample to absorb PCR inhibitors, the sample is mixed on a vortex for 1 min and subsequently centrifuged again for 6 min at maximum speed. Then, the supernatant is quantitatively transferred into a 1.5 ml tube. Each tube is pre-filled with 20 μl Proteinase K (20 mg/ml), and 300 μl of the supernatant are added to each tube and mixed. Subsequently, 300 μl AL-buffer are added to each tube and mixed on the vortex for 15 s. After a pre-incubation for 5 minutes at 70° C., the mixture is incubated for 10 min at 95° C. Then, 300 μl 100% ethanol are added and mixed thoroughly. The entire lysate is loaded in two steps onto the columns which are placed in 2 ml vials, followed by a centrifugation for 1 min at 20,000×g. The flow-through is discarded and columns are transferred into a new vial. For the washing step, 500 μl AW1 are given onto the columns, followed by centrifugation for 1 min at 20,000×g. The flow-through is discarded and columns are transferred into a new vial. For the washing step, 500 μl AW2 are given onto the columns, followed by centrifugation for 3 min at 20,000×g. The flowthrough is discarded; columns are transferred into a new vial and centrifuged again for 1 min at 20,000×g. The flow-through is discarded and columns are transferred into a new 1.5 ml vial. The subsequent elution of the DNA is performed by giving 100 μl AE-buffer directly onto the column and incubation for 1 min at room temperature. After a further centrifugation for 1 min at 20,000×g, the DNA is obtained in the collected eluate and can directly be used for real-time PCR analysis or stored frozen at −20° C.

Alternatively, other kits can be used instead of the DNeasy® Stool mini Kit from Qiagen, e.g. the High Pure PCR Template Preparation Kit® from the company Roche Applied Science which is also performed according to the manufacturer's instructions. Alternatively, milk can be used as sample material, in this case the DNA isolation is carried out using a state-of-the-art technique.

b) Analysis of the Isolated DNA in a Real-Time PCR, Whereby Both Marker Sequences ISMav2 and F57 are Amplified and Quantified

In order to select oligonucleotides as primers, reference sequences ISMav2 (gene accession number AF286339) SEQ ID NO:9 and F57 (gene accession number X70277) SEQ ID NO:10 as well as the MAP-genome sequence of reference strain K10 (gene accession number AE016958) were used which are deposited in the NCBI gene database.

Oligonucleotides chosen according to the present invention bind to a particularly favorable gene segment and are optimally positioned to guarantee a selective coverage of both MAP-markers ISMav2 and F57.

The size of the amplicon (approx. 120 base pairs for ISMav2 and approx. 60 base pairs for F57) are optimally adapted for the demands of the real-time PCR technology which requires a product size of 50 to 150 base pairs according to scientific and technical standards.

The small product size is a main prerequisite in order to assure high sensitivity and specificity. The probes are chosen in a way that they bind to amplified DNA and produce, independently of the amount of bound DNA, signals which can be measured and quantified. The probes are preferably labeled with a fluorescent dye, allowing a measurement of their fluorescence.

TABLE 1

| | | Binding- | Number |
|---|---|---|---|
| Nomenclature | Nucleotide sequence | position | of bases |
| SEQ ID NO: 1 | 5′-CGG CAA AAT CGA GCA GTT TC-3′ | 1645-1665 | 20 |
| forward primer | | ISMav2 | |
| IS-Mav2 | | | |
| SEQ ID NO: 2 | 5′-TGA GCC GGT GTG ATC ATC TTT-3′ | 1786-1807 | 21 |
| reverse primer | | ISMav2 | |
| IS-Mav2 | | | |
| SEQ ID NO: 3 | FAM-CGC TGA GTT CCT TAG-MGB | 1678-1693 | 15 |
| TaqMan_{mgb}-probe | | ISMav2 | |
| ISMav2 | | | |
| SEQ ID NO: 4 | FAM-cGc tGa GtT cCt TaG-BHQ1 | 1678-1693 | 15 |
| LNA-probe | | ISMav2 | |
| ISMav2 | | | |
| SEQ ID NO: 5 | 5′-TAC GAG CAC GCA GGC ATT C-3′ | 244-263 | 19 |
| forward primer | | F57 | |
| F57 | | | |
| SEQ ID NO: 6 | 5′-CGG TCC AGT TCG CTG TCA T-3′ | 288-307 | 19 |
| reverse primer | | F57 | |
| F57 | | | |
| SEQ ID NO: 7 | VIC-CCT GAC CAC CCT TC-MGB | 268-282 | 14 |
| TaqMan_{mgb}-probe | | F57 | |
| F57 | | | |
| SEQ ID NO: 8 | Yakima Yellow-cCtgAcCacCctTc-BHQ1 | 268-282 | 14 |
| LNA-probe F57 | | F57 | |

Table 1 shows the nucleic acid sequences of primers according to the present invention for the analysis of DNA using real-time PCR, and the nucleic acid sequence of probes TaqMan_{mgb}-probe and LNA-probe, respectively.

Primers according to the present invention are nucleic acid molecules which bind and hybridize to the ISMav2- and F57-marker sequence, respectively. Thereby it is clear to the expert that also variant derivatives of these primers are included which result from deletions, substitutions and/or insertions and which preferably exhibit at least 80% homology to the primers as shown here.

Sequences of the primers according to the present invention for the analysis of DNA using real-time PCR and the nucleic acid sequence of probes TaqMan_{mgb}-probe and LNA-probe, respectively, are composed of the following nucleic acid sequences:

Comment for the nomenclature of the primer and probe combinations:
TaqMan® is a registered trademark of the company Hoffman-La Roche.
The term mgb/MGB stands for minor groove binder and denotes a method for probe design which is well known to the expert and which allows for a higher binding affinity for the target region and a selection of probes with a shorter base sequence.
LNA-probe stands for Locked Nucleic Acids (LNAs), these are integrated into double dye labeled probes. LNA-bases increase the melting temperature markedly, therefore double dye labeled probes are shorter than conventional DNA-probes (<50 base pairs). Shorter probes show a more pronounced ‘quenching effect’, a lower back ground noise, and are thus more sensitive.
FAM denotes a substance chosen for the fluorescent dye labeling of probes, which is alternatively also called fluorescein and is well known to the expert in this field.
VIC likewise denotes a substance chosen for the fluorescent dye labeling of probes which is well known to the expert. The use of other fluorophores meets the technical standard and is known to those skilled in the art.

According to the present invention, TaqMan_{mgb}-probes and LNA-probes, respectively, are each used in combination with the respective primer pair as Duplex-real-time PCR. In combination with an internal amplification control (IAK), the application is carried out as so-called triplex real-time PCR. As analytical equipment, for example the ABI 7000 Sequence Detection System (company Applied Biosystems, Darmstadt) is used, alternatively also other equipments of the company Applied Biosystems as well as other commonly used devices which utilize the TaqMan-principle (BioRad Laboratories, Stratagene), as well as the Light Cycler V.2.0 of the company Roche.

For the detection of the ISMav2 marker sequence, forward primer ISMav2 SEQ ID NO:1 and forward primer ISMav2 SEQ ID NO:2 are combined with TaqMan_{mgb}-probe ISMav2 SEQ ID NO:3, or alternatively forward primer ISMav2 SEQ ID NO:1 and reverse primer ISMav2 SEQ ID NO:2 with LNA-probe ISMav2. SEQ ID NO:4.

For the detection of the F57 marker sequence, forward primer F57 SEQ ID NO:5 and reverse primer F57 SEQ ID NO:6 are combined with TaqMan_{mgb}-probe F57 SEQ ID NO:7, or alternatively forward primer F57 SEQ ID NO:5 and reverse primer F57 SEQ ID NO:6 with LNA-probe F57 SEQ ID NO:8.

For the quantification, DNA of a MAP-strain (e.g. of the bovine strain “423”) is isolated and the DNA concentration is determined. A decadal dilution series of this DNA is used in the respective real-time PCR.

For this purpose, the concentration of the isolated DNA is determined via UV-spectroscopy. Subsequently, in the course of the configuration of the real time cycler used, the decadal dilution series is defined as standard and values determined by spectroscopy are manually entered in the operation software of the thermocycler as basis of calculation. The DNA content of the samples to be investigated is determined in relation to the standards which are used in the same PCR run.

The following master mix conditions (50 μl-assay) are preferably used:

| Components for the master mix | Assay (1x in μl) |
|---|---|
| TaqMan Universal PCR Master Mix (2x) | 25.0 |
| MgCl₂ | 0 |
| SEQ ID NO: 1 forward primer _{”}ISMav2“ | 1.5 |
| SEQ ID NO: 2 reverse primer _{”}ISMav2“ | 1.5 |
| TaqMan_{mgb} probe SEQ ID NO: 3 or | 2.5 |
| LNA-probe _{”}ISMav2“ SEQ ID NO: 4 | |
| SEQ ID NO: 5 forward primer _{”}F57“ | 1.5 |
| SEQ ID NO: 6 reverse primer _{”}F57“ | 1.5 |
| TaqMan_{mgb} probe SEQ ID NO: 7 or | 2.5 |
| LNA -probe _{”}F57“ SEQ ID NO: 8 | |
| Aqua dest. | 9.0 |
| Template-DNA | 5.0 |
| Total volume | 50 μl |

Template-DNA is e.g. DNA isolated according to step a) from a biological sample or a MAP-positive or MAP-negative control sample, respectively.

Reagents used here are included as complete formula in the TaqMan-Universal Mastermix (Fa. Applied Biosystems, Darmstadt). Alternatively, other reagent e.g. of the company Eurogentec or Qiagen are used.

Conditions of the thermocycler are chosen as follows:

Initial UNG-incubation at 50° C. for 2 min, activation of the polymerase e.g. Ampli-Taq Gold Polymerase (Applied Biosystems, Darmstadt) at 95° C. The analysis time is 50 cycles, followed by a denaturation at 95° C. for 15 s and the annealing step at 60° C. for 1 min. Each determination is alternatively performed in duplicates. Data evaluation comprises the determination of the threshold cycle (Cₜ-value) and quantitative MAP-DNA content [unit: ng/μl] in the sample investigated. This is carried out using methods which are known to those skilled in the art.

The final evaluation of the results is performed according to a procedure which is commonly used by the expert and which is shown exemplarily in FIG. 1 for the diagram of the specific amplification of MAP.

Electronic collection of data and documentation are carried out in line with diagnostic standards of quality management for the modern laboratory practice. FIG. 2 shows a melting curve analysis. FIGS. 3 and 4 show the efficiency of amplification.

The procedure was validated using results from 49 MAP-, 34 non-MAP-, 65 non-Mycobacterium reference and field strains.

One embodiment of the present invention concerns a simply manageable test kit for the specific and sensitive detection of MAP in tissue and fecal samples, comprising at least:

SEQ ID NO: 1 forward primer _{”}ISMav2“
SEQ ID NO: 2 reverse primer _{”}ISMav2“
SEQ ID NO: 3 TaqMan_{mgb} or
SEQ ID NO: 4 LNA-probe _{”}ISMav2“
SEQ ID NO: 5 forward primer _{”}F57“
SEQ ID NO: 6 reverse primer _{”}F57“
SEQ ID NO: 7 TaqMan_{mgb} or
SEQ ID NO: 8 LNA -probe _{”}F57“

The test kit for the specific detection of Mycobacterium avium ssp. paratuberculosis (MAP) in fecal, tissue and organ samples using real-time PCR contains oligonucleotides as primers for the amplification of MAP-DNA accordant to SEQ ID NO:1, SEQ ID NO:2 and SEQ ID NO:5, SEQ ID NO:6, which hybridize with gene sequences for ISMav2 and F57 of the MAP-genome. Alternatively, the test kit contains probes which bind to the amplified DNA and produce, dependent on the amount of bound DNA, signals which can be measured and quantified. These probes are preferably linked with a fluorescent dye and represent SEQ ID NO:3 and SEQ ID NO:7 or alternatively SEQ ID NO:4 and SEQ ID NO:8.

In a preferred embodiment, the test kit includes furthermore reagents for the isolation of total genomic DNA from feces, organ and tissue samples, whereby the reagents reduce the amount of PCR inhibitors and contain proteinase K.

In addition, the test alternatively includes an internal amplification control which contains a defined amount MAP-DNA.

The test kit however also includes the formulation as single detection system using the oligonucleotides for the detection of “ISMav2” or “F57”, as well as the embodiment as multiplex-PCR.

On the basis of the scientific background of the present invention as well as on the basis of common expert knowledge in this technical field, the manufacturer of the kit according to the present invention is acquainted with the production, formulation, and storage of the single components of the kit, e.g. buffers.

### FIGURES

FIG. 1

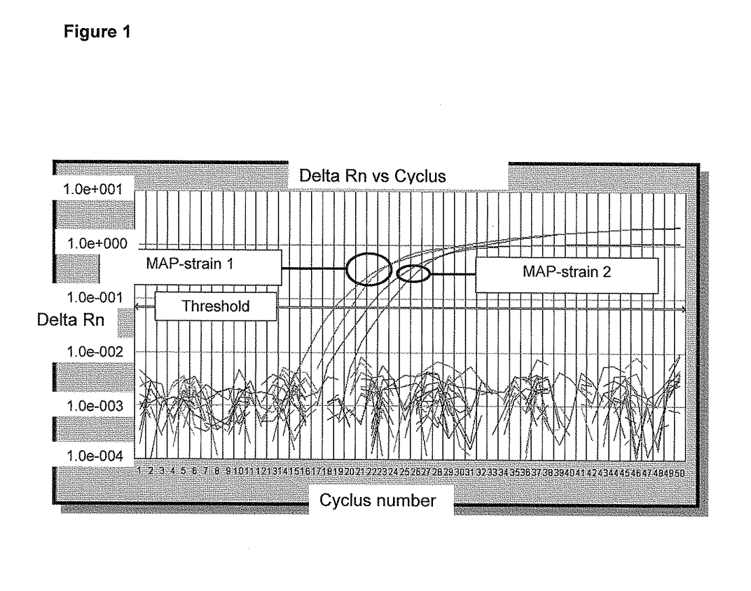

Specific amplification of two bovine MAP-strains MAP-strain 1 and MAP-strain 2 with markers “ISMav2” and “F57”

FIG. 2

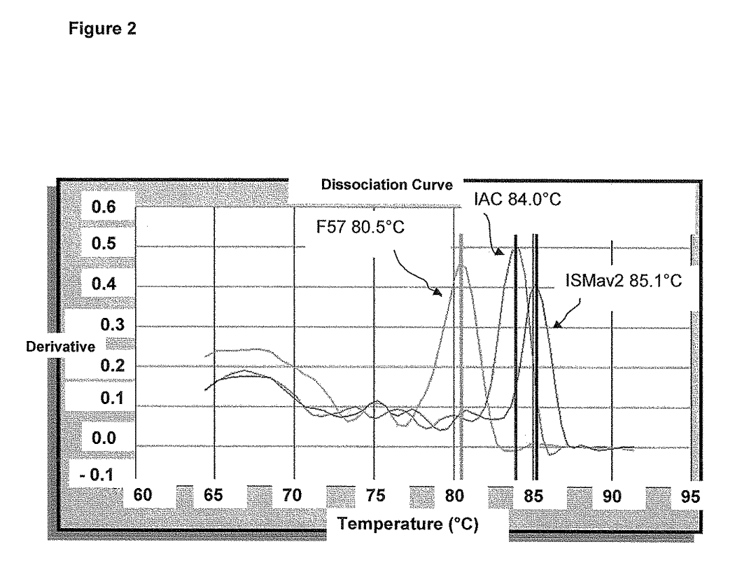

Dissociation curve and indication of melting point for the marker sequence “IS-Mav2” at 84.0° C., for the marker sequence “F57” at 80.5° C., and for the internal amplification control (IAK) at 85.1° C.

FIG. 3

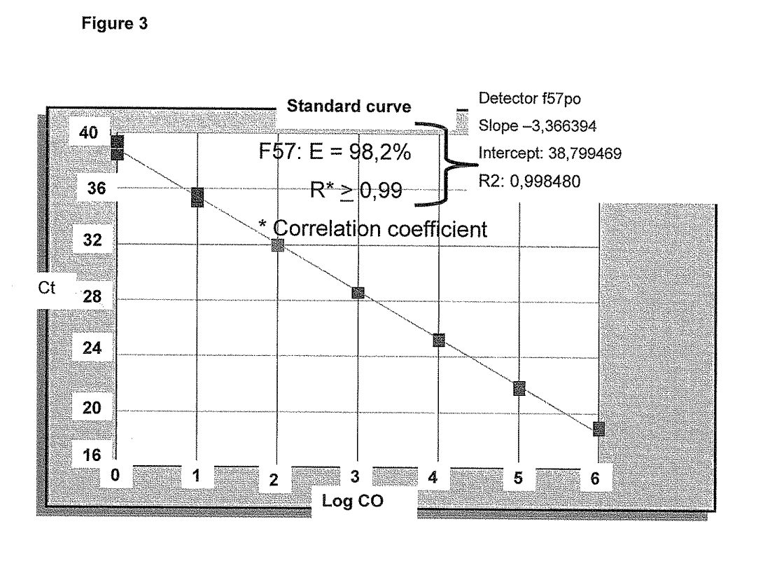

Amplification efficiency of “F57” in the triplex real-time PCR with the value: E=98.2%

FIG. 4

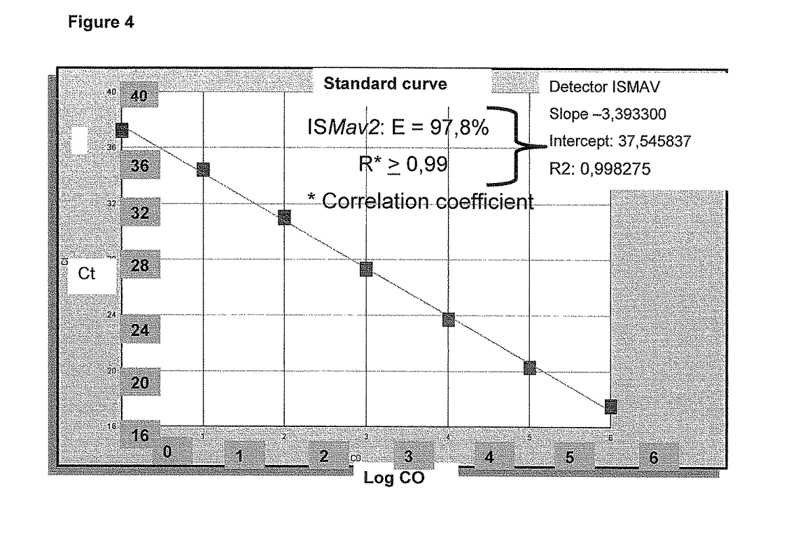

Amplification efficiency of “ISMav2” in a triplex real-time PCR with the value: E=97.8%

1. Oligonucleotides for the specific detection of Mycobacterium avium ssp. paratuberculosis (MAP) in fecal, tissue and organ samples using real-time-PCR, which is characterized in that
the oligonucleotides have the following nucleotide sequences:
SEQ ID NO: 1 forward primer ISMav2
5′-CGG CAA AAT CGA GCA GTT TC-3′
SEQ ID NO: 2 reverse primer ISMav2
5′-TGA GCC GGT GTG ATC ATC TTT-3′
SEQ ID NO: 5 forward primer F57
5′-TAC GAG CAC GCA GGC ATT C-3′
SEQ ID NO: 6 reverse primer F57
5′-CGG TCC AGT TCG CTG TCA T-3′

2. Procedure for the specific detection of Mycobacterium avium ssp. paratuberculosis (MAP) in fecal, tissue and organ samples using real-time-PCR, which is characterized in that
the marker sequences ISMAV2 SEQ ID NO:9 and F57 SEQ ID NO:10 of the MAP-genome are detected and the oligonucleotides SEQ ID NO:1, SEQ ID NO:2, SEQ ID NO:5 and SEQ ID NO:6 are used according to claim 1.

3. Procedure according to claim 2, which is characterized in that
the fecal, tissue and organ samples are derived from cattle, pigs, poultry, or humans.

4. Procedure according to claim 2, which is characterized in that
for the preparation of fecal, tissue and organ samples, reagents are used which reduce the amount of PCR inhibitors and contain proteinase K.

5. Procedure according to claim 2, which is characterized in that
DNA is isolated from the fecal, tissue and organ samples.

6. Procedure according to claim 2, which is characterized in that
the real-time PCR is performed in the presence of an internal amplification control.

7. Procedure according to claim 2, which is characterized in that
the internal amplification control contains a defined amount of MAP-DNA.

8. Procedure according to claim 2, which is characterized in that
the real-time PCR is carried out in the presence of probes, which bind to the amplified DNA and which produce, depending on the amount of bound DNA, signals that can be measured and quantified.

9. Procedure according to claim 8, which is characterized in that
as probe, TaqMan_{mgb} according to SEQ ID NO:3 and SEQ ID NO:7 or LNA-probe according to SEQ ID NO:4 and SEQ ID NO:8 is used.

10. Procedure according to claim 9, which is characterized in that
the probes are coupled to a fluorescent dye.

11. Test kit for the specific detection of Mycobacterium avium ssp. paratuberculosis (MAP) in fecal, tissue and organ samples using real-time PCR, which is characterized in that
the test kit includes oligonucleotides for the amplification of MAP-DNA according to claim 1, which hybridize to gene sequences for ISMav2 and F57 of the MAP-genome.

12. Test kit according to claim 11, which is characterized in that
probes are included which bind to amplified DNA and which, dependent on the amount of bound DNA, produce signals that can be measured and quantified.

13. Test kit according to claims 11, which is characterized in that
it includes probe TaqMan_{mgb} according to SEQ ID NO:3 and SEQ ID NO:7 or the LNA-probe according to SEQ ID NO:4 and SEQ ID NO:8.

14. Test kit according to claim 11, which is characterized in that
the probes are coupled to a fluorescent dye.

15. Test kit according to claim 11, which is characterized in that
it includes reagents for the isolation of total genomic DNA from fecal, organ and tissue samples, whereby the reagents reduce the amount of PCR inhibitors and contain proteinase K.

16. Test kit according to claim 11, which is characterized in that
it includes an internal amplification control, which contains a defined amount of MAP-DNA.
